(19) United States
(12) Patent Application Publication
(10) Pub. No.: US 2016/0033714 A1
(43) Pub. Date: Feb. 4, 2016

(54) BACKLIGHT MODULE AND DISPLAY DEVICE

(71) Applicants: BOE TECHNOLOGY GROUP CO., LTD., Beijing (CN); HEFEI BOE DISPLAY LIGHT CO., LTD., Anhui (CN)

(72) Inventor: Gang LIU, Beijing (CN)

(73) Assignees: BOE TECHNOLOGY GROUP CO., LTD., Beijing (CN); HEFEI BOE DISPLAYLIGHT CO., LTD., Anhui (CN)

(21) Appl. No.: 14/429,988

(22) PCT Filed: Jul. 31, 2014
(86) PCT No.: PCT/CN2014/083382

§ 371 Date: Mar. 20, 2015

(30) Foreign Application Priority Data
Jan. 28, 2014 (CN) 201410041979.7

(51) Int. Cl. F21V 8/00; B01J 19/12; F21K 99/00

(52) U.S. Cl. CPC G02B 6/0068; G02B 6/0073; F21K 9/30; B01J 19/123; G06F 2203/04103; F21Y 2113/005

(57) ABSTRACT

The present invention provides a backlight module and a display device, relates to the field of display technology, and can solve the problem of inconvenience in sterilization of an existing touch screen. The backlight module of the present invention comprises a plurality of light sources, and the plurality of light sources comprise ultraviolet light sources for sterilization and display light sources for display. The present invention particularly applies to a touch screen display device.

### FIELD OF THE INVENTION

The present invention relates to the field of display technology, and particularly relates to a backlight module and a display device.

### BACKGROUND OF THE INVENTION

Touch screen has been widely applied in various application fields of display, such as households, public places, office spaces, personal electronic products and the like. Touch screen technology creates a nourishing growing environment for bacteria while bringing unique experience to users. A British study shows that, the number of bacteria and viruses residing in a touch screen mobile phone should be 18 times as many as that adhering to a flush handle of a toilet in a lavatory. At present, people are paying increasing attention to environment and physical health, and such situation is worrisome.

Sterilization of an existing touch screen product is implemented mainly by adopting a separate sterilization device or a sterilization tissue for a touch screen. However, the following disadvantages exist in these two sterilization methods: 1, the separate sterilization device is inconvenient to carry, and especially when a touch screen product is contaminated in an unexpected situation, the separate sterilization device cannot treat the touch screen product immediately; 2, the sterilization tissues mostly contain components such as alcohol, disinfectant or the like, resulting in that the touch screen product, which has easy access to nose and mouth, may cause secondary contamination to human body after wiped by the sterilization tissue.

### SUMMARY OF THE INVENTION

The technical problem to be solved by the present invention includes providing a backlight module, which can perform sterilization treatment conveniently and quickly, in view of the above problems in sterilization of an existing touch screen product.

A technical solution adopted to solve the technical problem of the present invention is providing a backlight module, comprising a plurality of light sources, and the plurality of light sources comprise ultraviolet light sources for sterilization and display light sources for display.

The backlight module of the present invention includes ultraviolet light sources, and ultraviolet rays emitted from the ultraviolet light sources can kill bacteria, and thus, by applying the backlight module including the ultraviolet light sources in a display device, sterilization treatment can be performed on the display screen of the display device (especially, a display device with touch function). In this way, the problems that prompt sterilization treatment cannot be performed on the display screen when a separate sterilization device is adopted and secondary contamination may be caused to human body when a sterilization tissue is adopted to sterilize the display screen can be well solved. The backlight module of the present invention has simple structure, and strong applicability, and is convenient.

Preferably, the ultraviolet light sources are ultraviolet LED lamps, and the display light sources are display LED lamps.

Further preferably, the display LED lamps are arranged on a display LED light bar; and the ultraviolet LED lamps are arranged on an ultraviolet LED light bar.

Further preferably, both the display LED lamps and the ultraviolet LED lamps are arranged on a mixed LED light bar.

Preferably, the backlight module is a direct-type backlight module.

Preferably, the backlight module is a side-type backlight module.

Further preferably, the backlight module further comprises a light guide plate, which comprises four sides, the display LED light bar is arranged on one side of the light guide plate, and the ultraviolet LED light bar is arranged on at least one of the other three sides of the light guide plate.

Further preferably, the backlight module further comprises a light guide plate, which comprises four sides, and the mixed LED light bar is arranged on one side of the light guide plate.

Further preferably, the backlight module further comprises a time-delay module, which is connected to the ultraviolet light sources and is used for delaying lighting up the ultraviolet light sources by a set time when the backlight module is turned on.

A technical solution adopted to solve the technical problem of the present invention is providing a display device, comprising a display panel and the above backlight module.

Preferably, the display panel is a touch panel.

Preferably, the display panel comprises a substrate, which is made of borosilicate glass or quartz glass.

### BRIEF DESCRIPTION OF THE DRAWINGS

FIG. 1 is a front view of a configuration of a backlight module according to an embodiment of the present invention.

FIG. 2 is a side view of the backlight module in FIG. 1.

FIG. 3 is a front view of another configuration of a backlight module according to an embodiment of the present invention.

FIG. 4 is a side view of the backlight module in FIG. 3.

FIG. 5 is a front view of still another configuration of a backlight module according to an embodiment of the present invention.

FIG. 6 is a side view of the backlight module in FIG. 5.

FIG. 7 is a side view of a backlight module according to an embodiment of the present invention.

### DETAILED DESCRIPTION OF THE EMBODIMENTS

To enable those skilled in the art to better understand technical solutions of the present invention, the present invention will be further described in detail below in conjunction with the accompanying drawings and specific implementations.

An embodiment of the present invention provides a backlight module, which comprises a plurality of light sources. The plurality of light sources comprise ultraviolet light sources for sterilization and display light sources for display.

The light sources of the backlight module provided by the present embodiment include ultraviolet light sources. It can be understood that, ultraviolet rays emitted from the ultraviolet light sources can kill bacteria, and thus by applying the backlight module including the ultraviolet light sources in a display device, sterilization treatment can be performed on the display screen of the display device (especially, a display device with touch screen). In this way, the problems that prompt sterilization treatment cannot be performed on the display screen when a separate sterilization device is adopted and secondary contamination may be caused to human body when a sterilization tissue is adopted to sterilize the display screen can be well solved. The backlight module of the present embodiment has simple structure and strong applicability, and is convenient.

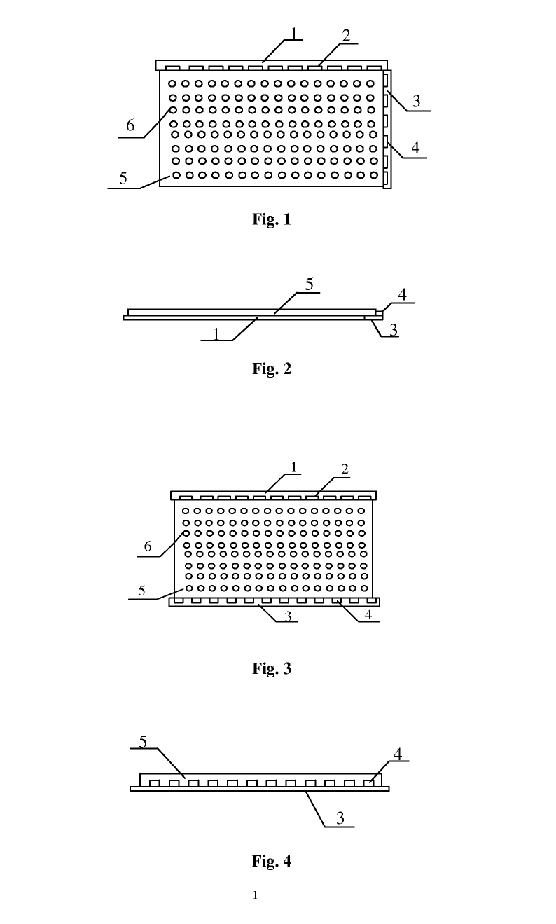

The ultraviolet light sources preferably are ultraviolet LED lamps 4, as shown in FIGS. 1 to 7, and needless to say, the ultraviolet light sources may be UVA lamps, ultraviolet high pressure mercury lamps, ultraviolet xenon lamps, ultraviolet metal halide lamps or the like, which can emit ultraviolet rays. The display light sources may be display LED lamps 2, specifically, may be white LED lamps, and of course may be LED lamps of other color.

Further preferably, the display LED lamps 2 are arranged on a display LED light bar 1; and the ultraviolet LED lamps 4 are arranged on an ultraviolet LED light bar 3, as shown in FIGS. 1 and 3.

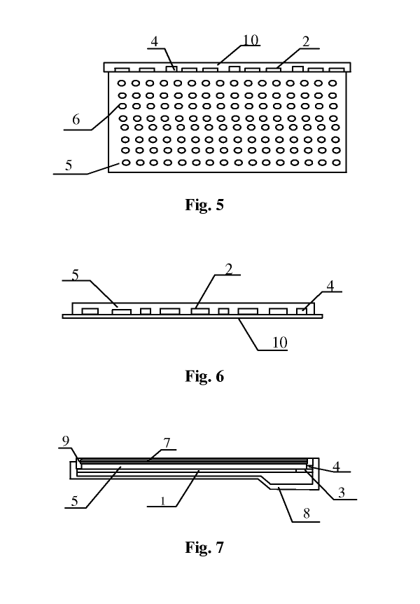

Alternatively, further preferably, both the display LED lamps 2 and the ultraviolet LED lamps 4 are arranged on a mixed LED light bar 10, as shown in FIG. 5.

Preferably, the backlight module of the present embodiment is a direct-type backlight module. In this case, the plurality of light sources including the ultraviolet light sources and the display light sources are configured to be a surface-like light source. An optical film layer 7 such as a diffusion film, a prism film, a composite film layer formed therefrom or the like is provided at a light-outgoing side of the surface-like light source. Specifically, the surface-like light source may be formed by arranging a plurality of the above display LED light bars 1 and a plurality of the above ultraviolet LED light bars 3 into a surface-like structure. Needless to say, the surface-like light source may also be formed by arranging a plurality of the above mixed LED light bars 10 into a surface-like structure.

In addition, the backlight module of the present embodiment may be a side-type backlight module. The backlight 5 may further comprise a light guide plate 5. It can be easily understood that, both the display light sources and the ultraviolet light sources are arranged at sides of the light guide plate 5. The light guide plate 5 comprises four sides, and then the side-type backlight module may adopt the following two structures.

In the first structure, the display LED light bar 1 is arranged on one side of the light guide plate 5, and the ultraviolet LED light bar 3 may be arranged on at least one of the remaining three sides of the light guide plate 5, as shown in FIGS. 1 and 3.

In the second structure, the display LED lamps 2 and the ultraviolet LED lamps 4 are arranged on the mixed LED light bar 10, and in this case, the mixed LED light bar 10 is arranged on one side of the light guide plate 5, as shown in FIG. 5.

It should be noted that, mesh points 6 in a matrix may be provided on the light guide plate 5, as shown in FIGS. 1, 3 and 5, and the mesh points 6 are used for better diffusing the light emitted from the ultraviolet LED lamps 4 and the display LED lamps 3. The light guide plate is preferably made of PMMA (polymethyl methacrylate); the optical film layer 7 is preferably made of PET (polyethylene glycol terephthalate). As shown in FIG. 7, the backlight module further comprises components such as sealant 9, back plate 8 and the like, which are similar to those of a conventional backlight module, and are not described herein. The optical film layer 7 is arranged at the light-outgoing side of the light guide plate 5. The optical film layer 7 may be a diffusion film, a prism film, a composite film layer formed from both, or the like.

In order to use the backlight module in a healthier and safer way, the present embodiment further comprises a time-delay module, which is connected to the ultraviolet light sources and is used for delaying lighting up the ultraviolet light sources by a set time (the set time ranges, for example, from 10 seconds to 20 seconds approximately, and can be specifically set according to specific conditions) when the backlight module is turned on. After the ultraviolet light sources are lit up, the user stays away from the backlight module (i.e., the display device) so as to avoid harm to the user caused by the ultraviolet rays emitted from the ultraviolet light sources. It can be understood by a person skilled in the art that, the time-delay module may be a simple time-delay circuit, for example, the time-delay circuit may be formed by connecting a time-delay chip (e.g., a 555 timer) with a controller, a delay time is set through a software built in the time-delay chip, and the ultraviolet light sources are lit up under the control of the controller. Specific working principle thereof is not described in detail herein.

It should be noted that, the backlight module in the present embodiment further comprises a driving unit used for driving the display light sources to emit light, and needless to say, the driving unit can also be used for driving the ultraviolet light sources to emit light.

An embodiment of the present invention also provides a display device comprising a display panel and the backlight module according to the above embodiment of the present invention. The display device may be any product or component with display function, such as a mobile phone, a tablet computer, a television, a monitor, a notebook computer, a digital photo frame, a navigator or the like.

Since the display device of the present embodiment comprises the backlight module according to the above embodiment of the present invention and a display panel, sterilization treatment can be performed quickly and safely.

Since a touch panel is frequently touched by a user's finger, and consequently the touch panel is contaminated, the above backlight module is more applicable to a touch display device, and the display panel is preferably a touch panel.

Preferably, the display panel provided by the present embodiment comprises a substrate, which is preferably made of borosilicate glass or quartz glass. Needless to say, the substrate may be made of other transparent material.

Needless to say, the display device provided by the present embodiment may also comprise other conventional structures such as a power supply unit, a display driving unit and the like.

It can be understood that, the above implementations are merely exemplary implementations used for explaining the principle of the present invention, but the present invention is not limited thereto. For those skilled in the art, various modifications and improvements may be made without departing from the spirit and essence of the present invention, and these modifications and improvements are also deemed as falling within the protection scope of the present invention.

1-20. (canceled)

21. A backlight module, comprising a plurality of light sources, wherein, the plurality of light sources comprise ultraviolet light sources for sterilization and display light sources for display.

22. The backlight module according to claim 21, wherein, the ultraviolet light sources are ultraviolet LED lamps, and the display light sources are display LED lamps.

23. The backlight module according to claim 22, wherein,
the display LED lamps are arranged on a display LED light bar; and
the ultraviolet LED lamps are arranged on an ultraviolet LED light bar.

24. The backlight module according to claim 22, wherein, both the display LED lamps and the ultraviolet LED lamps are arranged on a mixed LED light bar.

25. The backlight module according to claim 21, wherein, the backlight module is a direct-type backlight module.

26. The backlight module according to claim 21, wherein, the backlight module is a side-type backlight module.

27. The backlight module according to claim 23, wherein, the backlight module is a side-type backlight module.

28. The backlight module according to claim 24, wherein, the backlight module is a side-type backlight module.

29. The backlight module according to claim 27, further comprising a light guide plate, which comprises four sides, wherein, the display LED light bar is arranged on one side of the light guide plate, and the ultraviolet LED light bar is arranged on at least one of the other three sides of the light guide plate.

30. The backlight module according to claim 28, further comprising a light guide plate, which comprises four sides, wherein, the mixed LED light bar is arranged on one side of the light guide plate.

31. The backlight module according to claim 21, further comprising a time-delay module, which is connected to the ultraviolet light sources and is used for delaying lighting up the ultraviolet light sources by a set time when the backlight module is turned on.

32. The backlight module according to claim 22, further comprising a time-delay module, which is connected to the ultraviolet light sources and is used for delaying lighting up the ultraviolet light sources by a set time when the backlight module is turned on.

33. The backlight module according to claim 23, further comprising a time-delay module, which is connected to the ultraviolet light sources and is used for delaying lighting up the ultraviolet light sources by a set time when the backlight module is turned on.

34. The backlight module according to claim 24, further comprising a time-delay module, which is connected to the ultraviolet light sources and is used for delaying lighting up the ultraviolet light sources by a set time when the backlight module is turned on.

35. A display device, comprising a display panel and a backlight module, wherein, the backlight module comprises a plurality of light sources, and the plurality of light sources comprise ultraviolet light sources for sterilization and display light sources for display.

36. The display device according to claim 35, wherein, the display panel is a touch panel.

37. The display device according to claim 35, wherein, the display panel comprises a substrate, which is made of borosilicate glass or quartz glass.

38. The display device according to claim 35, wherein, the ultraviolet light sources are ultraviolet LED lamps, and the display light sources are display LED lamps.

39. The display device according to claim 38, wherein,
the display LED lamps are arranged on a display LED light bar; and
the ultraviolet LED lamps are arranged on an ultraviolet LED light bar.

40. The display device according to claim 38, wherein, both the display LED lamps and the ultraviolet LED lamps are arranged on a mixed LED light bar.
